(19) United States
(12) Patent Application Publication
(10) Pub. No.: US 2022/0208307 A1
(43) Pub. Date: Jun. 30, 2022

(54) CLASSIFYING CONDITIONS OF NATIVE ORGANS USING MODELS TRAINED ON CONDITIONS OF TRANSPLANTED ORGANS

(71) Applicant: TRANSCRIPTOME SCIENCES INC., Edmonton (CA)

(72) Inventor: Phillip Halloran, Edmonton (CA)

(21) Appl. No.: 17/602,472

(22) PCT Filed: Apr. 8, 2020
(86) PCT No.: PCT/IB2020/053368

§ 371 Date: Oct. 8, 2021

Related U.S. Application Data
(60) Provisional application No. 62/831,559, filed on Apr. 9, 2019.

(51) Int. Cl. G16B 40/00; G16H 50/20; G06N 3/08; G06N 20/20; G16B 40/30; G16B 25/10

(52) U.S. Cl. CPC G16B 40/00; G16H 50/20; G06N 3/08; A61B 10/0233; G16B 40/30; G16B 25/10; G06N 20/20

(57) ABSTRACT

This document provides machine learning and classification systems that can receive training data that includes expression profiles for tissue samples from non-native (transplanted) organs, and can generate one or more machine learning models configured to classify expression profiles for tissue samples from native (non-transplanted) organs

### CROSS-REFERENCE TO RELATED APPLICATION

This application claims the benefit of U.S. Provisional Ser. No. 62/831,559 filed Apr. 9, 2019. This disclosure of the prior application is considered part of (and is incorporated by reference in) the disclosure of this application.

### BACKGROUND

### 1. Technical Field

Molecular microscope diagnostic systems can be used for histology-independent assessments of biopsies from various transplanted organs, based on their expression of rejection-associated transcripts. The diagnostic systems can include using machine learning techniques for identifying various archetypes in biopsy data. Each archetype can correspond to a different type of rejection of a transplanted organ.

### 2. Background Information

Machine-learning involves the analysis of data samples to identify features and patterns in the data samples that can be employed to perform tasks such as classification and prediction without explicit instructions. Some machine-learning techniques determine a model for performing the desired task, such as a neural network, a regression model, a decision tree, a support vector machine, and a naïve Bayes machine.

### SUMMARY

This document describes a machine learning and classification system that can receive training data that includes expression profiles for tissue samples from non-native (transplanted) organs, and can generate one or more machine learning models configured to classify expression profiles for tissue samples from native (non-transplanted) organs. In some cases, the machine learning model(s) may be trained at least in part on training samples that describe expression profiles for tissue from transplanted organs of a different type than a native (non-transplanted) organ being classified. Possible classifications which may be applied by the machine learning model(s) include one or more classifications that represent a normal condition of a native (non-transplanted) organ, and one or more classifications that represent an abnormal condition of the organ. Classifications may be output and stored in a record associated with the organ or patient. In response to a native (non-transplanted) organ being classified as having an abnormal condition, a suitable alert can be generated for presentation to the patient or to a healthcare provider. Based on an assessment of the healthcare provider of whether the classification is accurate, the machine learning model(s) may be updated.

### BRIEF DESCRIPTION OF DRAWINGS

FIG. 1 depicts an example system that can classify conditions of native organs using machine learning models trained on conditions of transplanted organs.

FIG. 2 is a flowchart of an example process for training one or more machine learning models on expression profiles from training samples.

FIG. 3 is a flowchart of an example process for assessing a condition of a native organ based at least in part on a classification determined by a machine learning model.

FIG. 4A shows an example representation of an expression profile used to train a machine learning model.

FIG. 4B shows an example clustering of expression profile data.

FIG. 4C shows an example classification using a machine learning model.

FIG. 5 shows an example of a computing device and a mobile computing device that can be used to implement at least some of the techniques described herein.

The details of one or more implementations are set forth in the accompanying drawings and the description below. Other features, objects, and advantages will be apparent from the description and drawings, and from the claims.

### DETAILED DESCRIPTION

A machine learning and classification system is described herein that can receive training data that includes expression profiles for tissue samples from non-native (transplanted) organs, and can generate machine learning models configured to classify an expression profile for a tissue sample from a native (non-transplanted) organ as indicating a normal condition of the organ, or one or more abnormal conditions. Abnormal conditions, for example, can include one or more rejection states (e.g., T cell-mediated rejection (TCMR), antibody-mediated rejection (ABMR), or other rejection states), and one or more injury states. By separating injury states from rejection states, and because injury features are universal to expression profiles for tissue samples from many different types of organs, an injury phenotype of a native (non-transplanted) organ biopsy can be analyzed and compared to a reference set of normal and abnormal non-native (transplanted) biopsies. Such a comparison can create valuable information about a state of a native (non-transplanted) organ, because it can be compared using thousands of readily available expression profiles from injured organs that happen to be transplanted organs. In contrast, a reference set of native (non-transplanted) biopsies may not be as readily available for at least some organ types (e.g., the heart) due to difficulties in obtaining native biopsies of such organs.

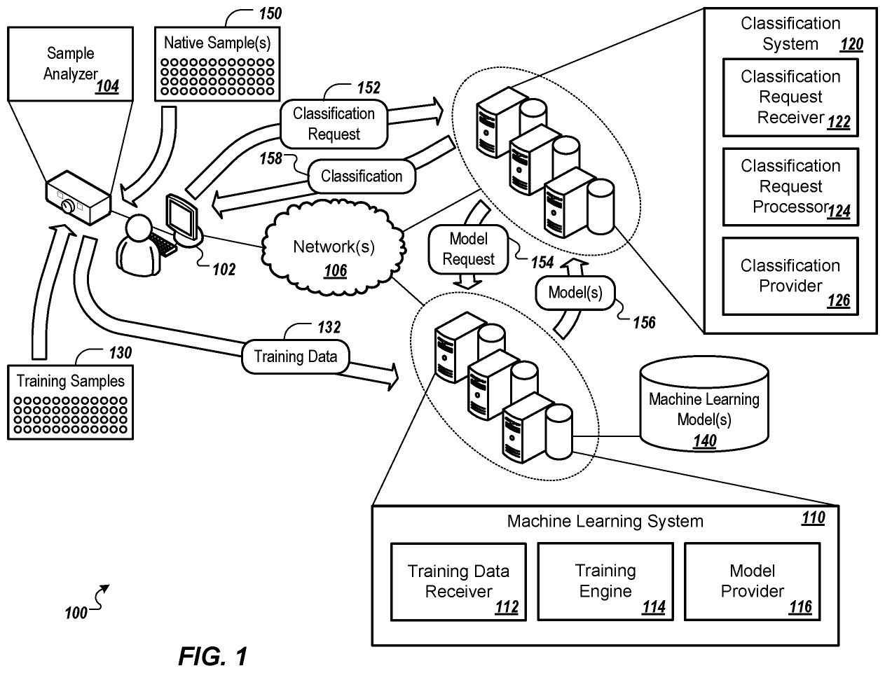

FIG. 1 depicts an example system 100 that can classify conditions of native organs using machine learning models trained on conditions of transplanted organs. In the depicted example, the system 100 includes a computing device 102 that communicates with a sample analyzer 104, a machine learning system 110, and a classification system 120 over one or more networks 106. In some examples, the computing device 102 may represent various forms of data processing devices including, but not limited to, a desktop computer, a laptop computer, a tablet computer, a handheld computer, or a combination of two or more of these data processing devices or other data processing devices. As discussed in further detail herein, the computing device 102 can interact with software executed by the sample analyzer 104, the machine learning system 110, and the classification system 120.

In some implementations, the system 100 may be a distributed client/server system that spans one or more networks such as network(s) 106. The network(s) 106, for example, can be a large computer network, such as a local area network (LAN), wide area network (WAN), the Internet, a cellular network, or a combination thereof connecting any number of mobile clients, fixed clients, and servers. In some implementations, each client (e.g., computing device 102) may communicate with the machine learning system 110 and the classification system 120 through a virtual private network (VPN), Secure Shell (SSH) tunnel, or other secure network connection. In some implementations, the network(s) 106 may include a wireless service network, and may include the Public Switched Telephone Network (PSTN). In some implementations, the network 104 may include a corporate network (e.g., an intranet) and one or more wireless access points.

In some implementations, the sample analyzer 104 may include software and hardware apparatus for analyzing biological sample material. In some cases, the sample analyzer 104, for example, can include one or more microarrays and a microarray reader or other suitable analysis hardware to identify ribonucleic acid (RNA) such as mRNA of collected tissue samples. For example, the sample analyzer 104 can perform molecular examination of one or more biopsied tissue samples, can measure the presence, absence, and/or amount of RNA (e.g., mRNA) for the samples, and can generate and provide transcript expression data that corresponds to the measured RNA (e.g., mRNA) for the samples. Examples of microarrays that can be used as described herein include, without limitation, PrimeView Human Gene Expression Array, PrimeView Human Genome U219 Array Plate, PrimeView Human Genome U219 Array Strip, and GeneChip Human Genome U133 Plus 2.0 Array. In some cases, the sample analyzer 104, for example, can use Next Generation Sequencing (e.g., RNAseq) to sequence DNA and RNA.

In some implementations, the machine learning system 110, the classification system 120, or both may include one or more servers and databases. In some examples, the one or more servers may include various forms of servers including, but not limited to application servers, web servers, or network servers. For example, the one or more servers can be application servers that execute software accessed by the computing device 102. In some implementations, a user may invoke applications available from the one or more servers in a user-interface application (e.g., a client application, a web browser, or another user-interface application) running on the computing device 102. The application, for example, can access data from one or more repository resources (e.g., databases and/or file systems) of the machine learning system 110 and/or the classification system 120. In some implementations, the machine learning system 110 and the classification system 120 may be executed using the same one or more servers and databases. In some implementations, the machine learning system 110 and the classification system 120 may be executed using different servers and/or databases.

In some implementations, the machine learning system 110, the classification system 120, or both may be executed by the computing device 102. In some examples, the computing device 102 may execute software and invoke applications locally available to the device, and the applications may access data from one or more repository resources locally available to the device. For example, the computing device 102 can interact with software executed by the same device 102, with storage occurring on the device 102.

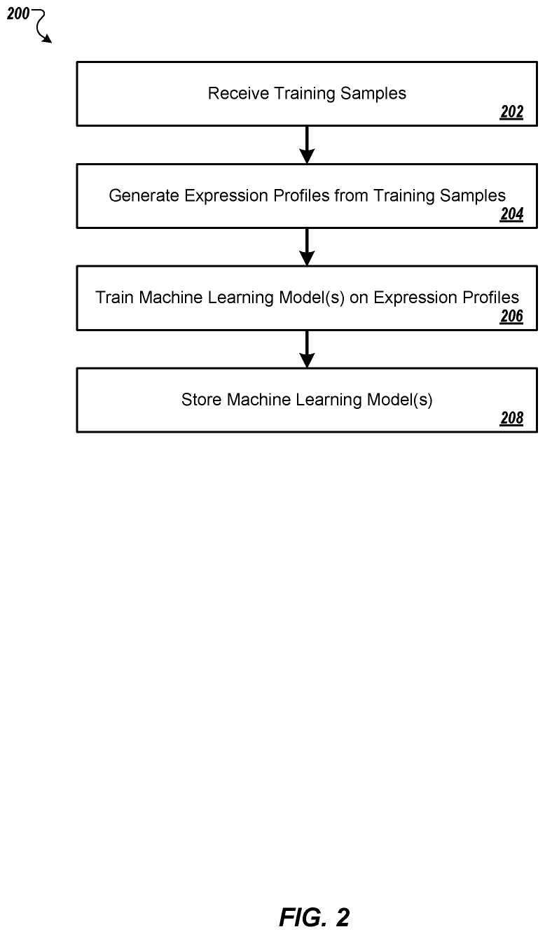

In some implementations, the machine learning system 110, the classification system 120, or both may include various components (e.g., software applications, services, modules, objects, or other suitable components), which may be combined or separate, and may be co-located or distributed. In the present example, the machine learning system 110 includes a training data receiver 112, a training engine 114, and a model provider 116. In the present example, the classification system 120 includes a classification request receiver 122, a classification request processor 124, and a classification provider 126. Operations of the machine learning system 110, the classification system 120, and their respective components are described in further detail below with respect to FIGS. 2, 3, and 4A-C.

Referring now to FIG. 2, a flowchart of an example process 200 for training one or more machine learning models on expression profiles from training samples is shown. In some implementations, the example process 200 may be provided by one or more computer-executable programs executed using one or more computing devices. The example process 200 can be implemented, for example, by the system 100 (shown in FIG. 1).

Training samples are received (202). For example, the sample analyzer 104 can receive training samples 130 that include, at least in part, tissue from non-native organs of a population of mammals, the organs having been transplanted into and hosted by the mammals (e.g., humans) for a period of time (e.g., a day, a week, a month, a year, or another suitable time period) before the samples 130 are collected. The population of mammals, for example, can include one or more of humans, monkeys, mice, rats, rabbits, dogs, cats, or other suitable types of mammals. The organs, for example, can include one or more of hearts, kidneys, livers, pancreases, lungs, or other suitable types of organs. Techniques for collecting the training samples 130, for example, can include one or more techniques for removing a small biological sample from an organ (e.g., a cluster of cells), a portion of an organ, or an entire organ, such techniques including a needle biopsy, an endoscopic biopsy, a vacuum-assisted biopsy, a surgical biopsy, or other suitable techniques.

Expression profiles (e.g., polypeptide expression profiles and/or RNA expression profiles) are generated from the training samples (204). In some implementations, collected training samples may be processed and analyzed using a suitable mRNA measurement platform (e.g., a DNA microarray or biochip), including a collection of microscopic DNA spots attached to a solid surface (e.g., glass, plastic, silicon, or another suitable surface). A DNA microarray or biochip, for example, can be used to simultaneously measure RNA expression levels of many genes (e.g., at least ten thousand genes, at least twenty thousand genes, at least thirty thousand genes, or another suitable number of genes). For example, the sample analyzer 104 can perform molecular examination of the received training samples 130 (e.g., using a microarray reader or other suitable analysis hardware), can measure the presence, absence, and/or amount of RNA (e.g., mRNA levels) for the samples 130, and can generate and provide training data 132 that includes expression profiles that corresponds to the measured RNA (e.g., mRNA) for the samples 130.

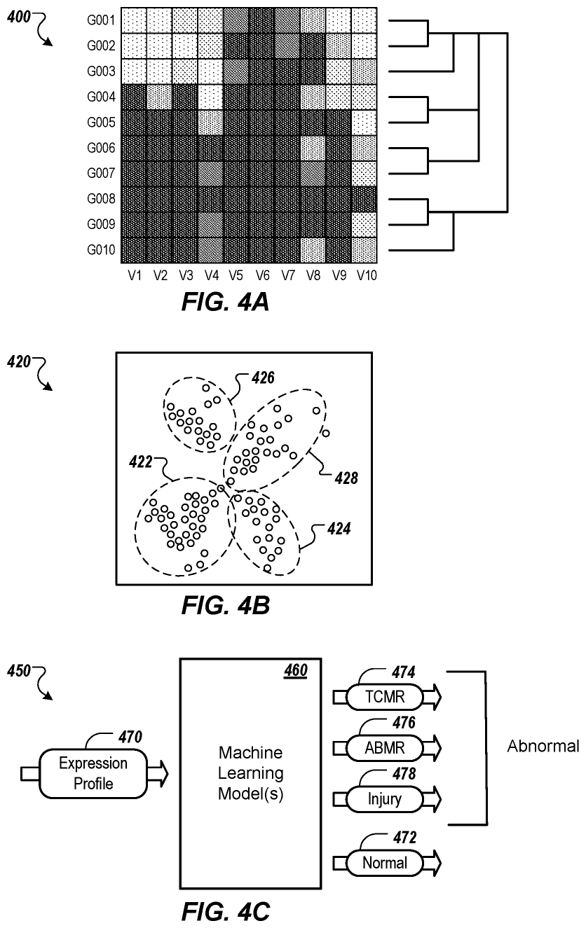

Referring now to FIG. 4A, an example representation of an expression profile 400 used to train a machine learning model is shown. In general, expression profiling can measure the presence, absence, and/or amount of expression for thousands of genes at once (e.g., at least ten thousand genes, at least twenty thousand genes, at least thirty thousand genes, or another suitable number of genes), and can be used to describe cellular function of a collected tissue sample at a given point in time, the cellular function possibly being influenced by environmental conditions associated with the tissue sample. In the present example, the expression profile 400 is shown as a heat map of gene expression values that indicate how conditions influence production of RNA (e.g., mRNA) for a set of genes. For example, the expression profile 400 can correspond to measured RNA (e.g., mRNA) values for one of the training samples 130, and can be included in the training data 132.

Referring again to FIG. 2, one or more machine learning models are trained on the expression profiles (206). For example, the machine learning system 110 can use the training data receiver 112 to receive the training data 132 (e.g., including expression profiles that correspond to measured RNA such as mRNA for the training samples 130), and can use the training engine 114 to train one or more machine learning models on the received data. In some implementations, the one or more machine learning models may be configured to classify the condition of a given organ by selecting a classification from a plurality of possible classifications, the plurality of possible classifications including a first classification or a first group of classifications that represent a normal condition of the given organ, and a second classification or a second group of classifications that represent an abnormal condition of the given organ. The first classification (or first group of classifications) that represent a normal condition of the given organ, for example, may be applied to the given organ to indicate that the organ is healthy (or not in a state of injury or not in a state of inflammation). The second classification (or second group of classifications) that represent an abnormal condition of the given organ, for example, may be applied to the organ to indicate that the organ is unhealthy. Abnormal conditions, for example, can include one or more rejection states (e.g., T cell-mediated rejection (TCMR), antibody-mediated rejection (ABMR), or other rejection states), and/or one or more injury states (e.g., based on response to wounding (RTW) or other conditions such as inflammation).

In some implementations, one or more machine learning models may be trained using at least one unsupervised machine learning technique. For example, the machine learning system 110 can use the training engine 114 to analyze expression profiles included in the received training data 132, the data having not been previously labelled, classified, or categorized. The unsupervised machine learning technique, for example, can identify commonalities in the training data 132 data and can learn relationships between data elements.

In some implementations, one or more machine learning models may be trained using at least one clustering technique. In general, clustering techniques may include various unsupervised machine learning techniques for grouping training data 132 such that data in a same cluster are more similar to each other than data in other clusters. Clustering techniques used by the training engine 114, for example, can include hierarchical clustering, k-means clustering, mixture models, or other suitable clustering techniques.

In some implementations, one or more machine learning models may be trained using at least one principal component analysis (PCA) technique. In general, PCA techniques may include various statistical procedures to perform exploratory data analysis and to generate predictive models. PCA techniques used by the training engine 114, for example, can include normalizing the training data 132, and generating PCA scores for the expression profiles in the training data.

In some implementations, one or more machine learning models may be trained using at least one neural network. In general, neural networks include an interconnected group of artificial neurons or nodes, which can be used for predictive modeling applications. For example, the training engine 114 can use a neural network to identify patterns in the training data 132.

In some implementations, an ensemble of different machine learning algorithms may be used. For example, the ensemble of different machine learning algorithms may include one or more of unsupervised machine learning techniques, semi-supervised machine learning techniques, supervised machine learning techniques, clustering techniques, principal component analysis (PCA) techniques, neural networks, regression analysis techniques, or other suitable machine learning and/or data analysis techniques. Using the ensemble of different machine learning algorithms may include, for each machine learning algorithm in the ensemble, determining a classification score for received sample data, and combining the classification scores to determine an aggregate score. In general, by using an ensemble of different machine learning algorithms, stable classifications may be determined for sample data, without undue reliance on any single technique.

Referring now to FIG. 4B, an example clustering 420 of expression profile data is shown. The example clustering 420, for example, can result from the training engine 114 applying one or more machine learning models to analyze expression profiles in the training data 132. In the present example, the clustering 420 includes clusters 422, 424, 426, and 428, each cluster corresponding to a different possible classification or archetype for an expression profile of a tissue sample. Cluster 422, for example, groups expression profiles of tissue samples associated with scores that indicate a normal condition of an organ. Clusters 424, 426, and 428, for example, group expression profiles of tissue samples associated with scores that indicate an abnormal condition of an organ. For example, cluster 424 can be associated with T cell-mediated rejections (TCMR) of organs, cluster 426 can be associated with antibody-mediated rejections (ABMR) of organs, and cluster 428 can be associated with an injury state of an organ.

Referring again to FIG. 2, one or more machine learning models are stored (208). For example, the machine learning system 110 can store the one or more machine learning models in a data store 140 of machine learning models. The data store 140, for example, can include one or more databases, file systems, and/or cached data sources, and can provide stored machine learning models upon request by various computing devices and/or systems.

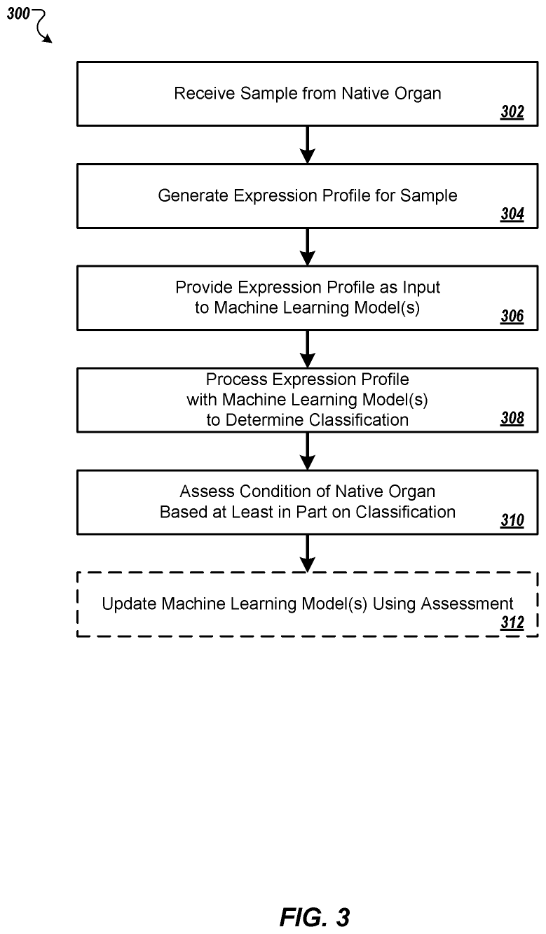

Referring now to FIG. 3, a flowchart of an example process 300 for assessing a condition of a native organ based at least in part on a classification determined by a machine learning model is shown. In some implementations, the example process 300 may be provided by one or more computer-executable programs executed using one or more computing devices. The example process 300 can be implemented, for example, by the system 100 (shown in FIG. 1).

A sample is received from a native organ of a mammal (302). For example, the sample analyzer 104 can receive one or more native samples 150 from the mammal, the native samples being tissue samples from an organ that is native to and has not been transplanted into the mammal. The mammal, for example, can be a human, a monkey, a mouse, a rat, a dog, a cat, or another suitable type of mammal. The organ from which the native sample 150 is obtained, for example, can be a heart, a kidney, a liver, a pancreas, a lung, or another suitable type of organ. Similar to techniques for collecting the training samples 130, for example, techniques for collecting the one or more native samples 150 can include one or more techniques for removing a small biological sample from an organ (e.g., a cluster of cells), a portion of an organ, or an entire organ, such techniques including a needle biopsy, an endoscopic biopsy, a vacuum-assisted biopsy, a surgical biopsy, or other suitable techniques.

An expression profile is generated for the sample from the native organ (304). Similar to techniques for generating expression profiles for the training samples 130, for example, the sample analyzer 104 can perform molecular examination of the native sample 150, can measure the presence, absence, and/or amount of polypeptides and/or RNA for the sample 150, and can generate and provide data that represents an expression profile (e.g., a polypeptide expression profile and/or an RNA expression profile) that corresponds to the measured polypeptides and/or RNA (e.g., mRNA) for the sample 150. For example, the sample analyzer 104 can process and analyze the native sample 150 using a suitable polypeptide and/or RNA (e.g., mRNA) measurement platform (e.g., a DNA microarray or biochip), including a collection of microscopic DNA spots attached to a solid surface (e.g., glass, plastic, silicon, or another suitable surface). The DNA microarray or biochip, for example, can be used to simultaneously measure RNA expression levels of many genes included in the native sample 150 (e.g., at least ten thousand genes, at least twenty thousand genes, at least thirty thousand genes, or another suitable number of genes).

The expression profile is provided as input to one or more machine learning models (306). For example, the computing device 102 can provide a classification request 152 to the classification system 120, the classification request 152 including data that represents the expression profile for the native sample 150. The classification system 120, for example, can use the classification request receiver 122 to receive the classification request 152.

The expression profile is processed with the one or more machine learning models to determine a classification for the expression profile (308). For example, the classification system 120 can use the classification request processor 124 to process the received classification request 152. Processing the classification request 152, for example, can include the classification system 120 sending a model request 154 to the machine learning system 110, the model request 154 being for one or more machine learning models that have been previously trained using training data 132 that represent expression profiles from training samples 130. The training samples 130, for example, can include, at least in part, tissue from non-native organs of a population of mammals (e.g., humans), the organs having been transplanted into the mammals. In response to receiving the model request 154, for example, the machine learning system 110 can use the model provider 116 to retrieve one or more suitable machine learning models from the data store 140 of machine learning models, and to provide the one or more models 156 to the classification system 120. Based on the model(s) 156, for example, the classification system 120 can classify the expression profile for the native sample 150, and can use the classification provider 126 to provide a classification 158 of the expression profile to the computing device 102.

In some implementations, a machine learning model used to classify a native (non-transplanted) organ may have been trained at least in part on training samples that describe expression profiles for tissue from non-native (transplanted) organs of a different type than the native organ for which the input is obtained. For example, the training samples 130 may be obtained from non-native (transplanted) kidneys, whereas the native sample 150 may be obtained from a native (non-transplanted) heart, liver, pancreas, or lung. As another example, the training samples 130 may be obtained from non-native (transplanted) hearts, whereas the native sample 150 may be obtained from a native (non-transplanted) organ of a different type. As another example, the training samples 130 may be obtained from non-native (transplanted) livers, whereas the native sample 150 may be obtained from a native (non-transplanted) organ of a different type. As another example, the training samples 130 may be obtained from non-native (transplanted) pancreases, whereas the native sample 150 may be obtained from a native (non-transplanted) organ of a different type. As another example, the training samples 130 may be obtained from non-native (transplanted) lungs, whereas the native sample 150 may be obtained from a native (non-transplanted) organ of a different type. As another example, the training samples 130 may be obtained from both native (non-transplanted) organs and non-native (trans-planted) organs, and the training samples 130 may include samples obtained from organs all of the same type or of different types.

In some implementations, a machine learning model used to classify a native (non-transplanted) organ may have been trained at least in part on training samples that describe expression profiles for tissue from non-native (transplanted) organs from a different species of mammal than a mammal that hosts the native organ for which the input is obtained. For example, the training samples 130 may be obtained from organs that have been transplanted into non-human animals, whereas the native sample 150 may be obtained from a native (non-transplanted) organ of a human. As another example, the training samples 130 may be obtained from organs of a mammal of a first species (e.g., a pig or a cow), whereas the native sample 150 may be obtained from a native (non-transplanted) organ of a second species (e.g., a human). The training samples 130 may include samples that describe expression profiles for tissue from non-native (transplanted) organs of the first species and, optionally, samples that describe expression profiles for tissue from native (non-transplanted) organs of the first species. In some cases, the training samples 130 that describe the expression profiles for tissue of organs of the first species (e.g., a pig or cow) may include samples that describe the expression profiles for tissue of a transplanted xenograft.

Referring now to FIG. 4C, an example classification 450 using one or more machine learning models 460 is shown. For example, the machine learning model(s) 460 (e.g., similar to the model(s) 156, shown in FIG. 1) can be used by the classification system 120 (shown in FIG. 1) to process expression profile 470 (e.g., based on data included in the classification request 152, shown in FIG. 1). In the present example, the machine learning model(s) 460 can be configured to classify the expression profile 470 as representing a normal condition 472 of a native (non-transplanted) organ, or one or more abnormal conditions, including a T cell-mediated rejection (TCMR) condition 474, an antibody-mediated rejection (ABMR) condition 476, and/or an injury condition 478. In some implementations, a primary classification can be provided for a native (non-transplanted) organ. For example, the machine learning model(s) 460 can determine respective scores for each of a plurality of different conditions, and a condition having a highest score can be designated as the primary classification for the organ.

Referring again to FIG. 3, a condition of the native organ is assessed based at least in part on the classification (310). For example, the computing device 102 can output (e.g., using a display, a speaker, a printer, or another suitable output device) the classification 158 for the native (non-transplanted) organ corresponding to the native sample 150, and a condition of the organ can be assessed in view of the classification 158. In some implementations, in response to outputting the classification that indicates the condition of the native (non-transplanted) organ, the classification may be stored in a record associated with at least one of the native organ or the mammal. For example, the computing device 102 can provide an identifier for the native organ and/or the mammal (e.g., a human patient) to a medical record system (not shown), in association with the classification. In some implementations, in response to a classification indicating that the condition of the native (non-transplanted) organ is abnormal, an alert may be generated for presentation to the mammal (e.g., a human patient) or a healthcare provider, the alert indicating that the native organ has been classified as having an abnormal condition. For example, the computing device 102 can use one or more of its output devices to generate such an alert.

Optionally, one or more of the one or more machine learning models may be updated using an assessment (312). For example, a healthcare provider can perform one or more diagnostic techniques (e.g., histological techniques) to confirm that the classification 158 that indicates the condition of the native (non-transplanted) organ of the mammal was accurate, and the assessment can be used to update one or more of the machine learning model(s) 140. For example, the assessment can be used to label sample data used in semi-supervised machine learning and/or supervised machine learning models.

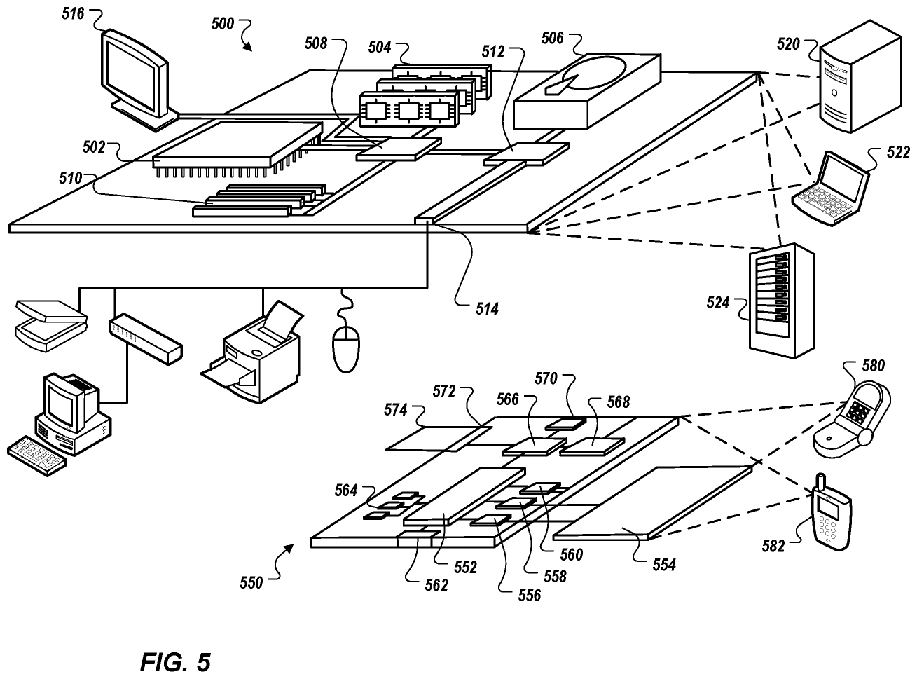

FIG. 5 shows an example of a computing device 500 and a mobile computing device 550 that can be used to implement the techniques described herein. The computing device 500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk.

The storage device 506 is capable of providing mass storage for the computing device 500. In some implementations, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. The computer program product may also contain instructions that, when executed, perform one or more methods, such as those described above. The computer program product can also be tangibly embodied in a computer- or machine-readable medium, such as the memory 504, the storage device 506, or memory on the processor 502.

The high-speed interface 508 manages bandwidth-intensive operations for the computing device 500, while the low-speed interface 512 manages lower bandwidth-intensive operations. Such allocation of functions is exemplary only. In some implementations, the high-speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device (not shown), such as a mobile computing device 550. Each of such devices may contain one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provided as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. The computer program product contains instructions that, when executed, perform one or more methods, such as those described above. The computer program product can be a computer- or machine-readable medium, such as the memory 564, the expansion memory 574, or memory on the processor 552. In some implementations, the computer program product can be received in a propagated signal, for example, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 568 using a radio-frequency. In addition, short-range communication may occur, such as using a Bluetooth, WiFi, or other such transceiver (not shown). In addition, a GPS (Global Positioning System) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in the figure. For example, it may be implemented as a cellular telephone 580. It may also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Although various implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

1. A computer-implemented method comprising:
obtaining an input that represents an expression profile for tissue from a native organ of a mammal;
processing the input with a machine learning model to determine a classification that indicates a condition of the native organ, wherein the model was trained at least in part on training samples that represented expression profiles for tissue from transplanted organs in a population of mammals, and the model is configured to classify the condition of a given organ by selecting the classification from a plurality of possible classifications, the plurality of possible classifications including a first classification or a first group of classifications that represent a normal condition of the given organ and a second classification or a second group of classifications that represent an abnormal condition of the given organ; and
outputting the classification that indicates the condition of the native organ of the mammal.

2. The computer-implemented method of claim 1, wherein the expression profile for the tissue from the native organ identifies messenger RNA (mRNA) expressed in the tissue.

3. The computer-implemented method of claim 2, wherein the expression profile for the tissue from the native organ measures an expression of mRNA for at least ten thousand genes, twenty thousand genes, or thirty thousand genes.

4. The computer-implemented method of claim 1, wherein the native organ is a heart, a kidney, a liver, a pancreas, or a lung of the mammal.

5. The computer-implemented method of claim 1, wherein the plurality of possible classifications from which the model is configured to select the classification of the given organ includes the second group of classifications that represent an abnormal condition of the given organ, the second group of classifications including classifications representing a T cell-mediated rejection (TCMR), an antibody-mediated rejection (ABMR), and an injury state.

6. The computer-implemented method of claim 1, wherein the model was trained using at least one unsupervised machine learning technique.

7. The computer-implemented method of claim 1, wherein the model was trained using at least one clustering technique.

8. The computer-implemented method of claim 1, wherein the model was trained using at least one principal component analysis (PCA) technique.

9. The computer-implemented method of claim 1, wherein the model was training using at least one neural network.

10. The computer-implemented method of claim 1, wherein the model includes an ensemble of different machine learning algorithms.

11. The computer-implemented method of claim 1, wherein the model was trained at least in part on training samples that describe expression profiles for tissue from transplanted organs of a different type than the native organ for which the input is obtained.

12. The computer-implemented method of claim 1, wherein the model was trained at least in part on training samples that describe expression profiles for tissue from transplanted organs of a same type as that of the native organ for which the input is obtained.

13. The computer-implemented method of claim 1, further comprising:
updating the machine learning model based at least in part on an assessment of a healthcare provider of whether the classification that indicates the condition of the native organ of the mammal was accurate.

14. The computer-implemented method of claim 1, further comprising:
in response to outputting the classification that indicates the condition of the native organ, storing the classification in a record associated with at least one of the native organ or the mammal.

15. The computer-implemented method of claim 1, further comprising:
in response to the classification indicating that the condition of the native organ is abnormal, generating an alert for presentation to the mammal or a healthcare provider that indicates that the native organ has been classified as having an abnormal condition.

16.-17. (canceled)

18. A method, comprising:
obtaining a tissue sample from a native organ of a mammal:
providing the tissue sample to a processor to obtain an expression profile for the tissue sample;
providing the expression profile for the tissue sample to a model that is configured to determine a classification that indicates a condition of the native organ, wherein the model was trained at least in part on training samples that describe represented expression profiles for tissue from transplanted organs in a population of mammals, and the model is configured to classify the condition of a given organ by selecting the classification from a plurality of possible classifications, the plurality of possible classifications including a first classification or a first group of classifications that represent a normal condition of the given organ and a second classification or a second group of classifications that represent an abnormal condition of the given organ;
obtaining, from the model, a classification of the condition for the native organ; and
assessing the condition of the native organ using the classification of the condition for the native organ.

19. A system, comprising:
one or more processors; and
a computer-readable storage device coupled to the one or more processors and having instructions stored thereon which, when executed by the one or more processors, cause the one or more processors to perform operations comprising:
obtaining an input that represents an expression profile for tissue from a native organ of a mammal;
processing the input with a machine learning model to determine a classification that indicates a condition of the native organ, wherein the model was trained at least in part on training samples that represented expression profiles for tissue from transplanted organs in a population of mammals, and the model is configured to classify the condition of a given organ by selecting the classification from a plurality of possible classifications, the plurality of possible classifications including a first classification or a first group of classifications that represent a normal condition of the given organ and a second classification or a second group of classifications that represent an abnormal condition of the given organ; and
outputting the classification that indicates the condition of the native organ of the mammal.
